(19) United States
(12) Patent Application Publication
(10) Pub. No.: US 2024/0424004 A1
(43) Pub. Date: Dec. 26, 2024

(54) COMBINATION OF TLR LIGANDS, COMPOUNDS LABELLING TUMORS FOR IMMUNE ATTACK, ANTI-CD40 ANTIBODIES AND INHIBITORS OF GLUTAMINE METABOLISM FOR TREATING CANCER

(71) Applicant: BIOCANIM A.S., Praha (CZ)

(72) Inventors: Jan ŽENKA, Caske Budejovice (CZ); Andrea FREJLACHOVÁ, Dynin (CZ); Radka LENCOVÁ, Dub (CZ); Ondrej UHER, Oracov (CZ)

(21) Appl. No.: 18/710,222

(22) PCT Filed: Nov. 3, 2022
(86) PCT No.: PCT/CZ2022/050115

§ 371 Date: May 15, 2024

Related U.S. Application Data
(60) Provisional application No. 63/279,212, filed on Nov. 15, 2021.

(51) Int. Cl. A61K 31/655; A61K 31/4355; A61K 31/713; A61K 31/736; A61K 31/739; A61P 35/00; C07K 16/28

(52) U.S. Cl. CPC A61K 31/655; A61K 31/4355; A61K 31/713; A61K 31/736; A61K 31/739; A61P 35/00; C07K 16/2878

(57) ABSTRACT

The object of the present invention is a pharmaceutical combination of active substances which comprises at least one TLR ligand, at least one compound that labels tumor cells as a target for immune cell attack and at least one anti-CD40 antibody, and further at least one inhibitor of glutamine metabolism. The use of the pharmaceutical combination is for cancer treatment, especially for solid tumor treatments as pancreatic adenocarcinoma.

### FIELD OF THE INVENTION

The present invention relates to a pharmaceutical combination and use thereof for the treatment of cancer, in particular for the treatment of solid tumors.

### BACKGROUND ART OF THE INVENTION

Tumor immune resistance is a very complex process consisting of multiple, commonly independent intracellular as well as microenvironmental mechanisms. Pancreatic adenocarcinoma is an example of tumor with strong immune resistance. Pancreatic adenocarcinoma has a unique tumor microenvironment, including a dense stroma, low mutation load, and immuno-suppressive mechanisms. The stromal environment creates a significant biophysical barrier to immune and other cells contributing to disease aggressiveness and enhanced drug resistance. The low mutation load and the immunosuppressive microenvironment prevent the recognition of tumor cells and suppress anti-tumor immunity. The poor clinical outcome thus calls for an urgent need to develop new innovative treatment approaches to bring hope to patients with this diagnosis.

Today, we are witnessing significant progress in the field of cancer immunotherapy. The most studied area is the use of checkpoint inhibitors. Attention is also focused on the use of CAR-T lymphocytes. In the field of checkpoint inhibitors, the focus is shifting from anti-CTLA-4 antibodies to PD-1/PD-L1 intervention and the combination of checkpoint inhibitors with other immuno-, chemo- and radio therapeutic approaches. However, the use of checkpoint inhibitors is effective only in about 20 percent of patients in a limited number of diagnoses. The main reason for the low effectiveness of checkpoint inhibitors is that the defence of tumors against immune attack is very complex and consists of a number of often independent mechanisms. Checkpoint inhibitors and CAR-T resolve only partial issues in this mosaic.

In previous work by the present inventors, an immunotherapy composition has been developed that includes TLR (Toll-like receptor) ligands and compounds that label tumor cells as a target for innate immune cell attack. This therapeutic mixture has shown good results in the treatment of melanoma (Caisová V, Vieru A, Kumžáková Z et al., Innate immunity-based cancer immunotherapy: B16-F10 murine melanoma model, BMC Cancer, p. 1-11, 2016; Caisová V, Uher O, Nedbalova P et al., Effective cancer immunotherapy based on combination of TLR agonists with stimulation of phagocytosis, Int Immunopharmacol., p. 86-96, 2018). In the treatment of very difficult-to-treat pancreatic adenocarcinoma and pheochromocytoma, it has been found to be very beneficial to enrich the therapeutic mixture with an agonist anti-CD40 antibody that promotes both innate and adaptive immunity (Caisová V, Li L, Gupta G et al., The significant reduction or complete eradication of subcutaneous and metastatic lesions in a pheochromocytoma mouse model after immunotherapy using mannan-BAM, TLR ligands, and-anti-CD40, Cancers, p 1-21, 2019).

This immunotherapy is very effective, but it has certain limits and drawbacks. It is difficult or impossible to completely eliminate large tumors and large metastases with this therapy. Therefore, further improvement of this immunotherapy was sought.

In the search for improvements in previously known immunotherapy, other therapeutic approaches compatible with this immunotherapy and acting synergistically have been sought. A suitable therapeutic approach has been very difficult to find because many therapeutic options, such as chemotherapy or radiotherapy, suppress the immune system, and thus their combination with immunotherapy to support the functions of innate immunity is counterproductive.

It is the object of the invention to develop a pharmaceutical combination for use in the treatment of cancer that would result in the complete elimination of advanced subcutaneous tumor if used in treatment and furthermore would provide long-term immune memory.

### DISCLOSURE OF THE INVENTION

The object of the invention is achieved by development of a pharmaceutical combination for use in the treatment of cancer of active substances for immunotherapy, said combination comprising at least one TLR (Toll-like receptor) ligand, at least one compound that labels tumor cells as a target for immune cell attack, and at least one anti-CD40 antibody, and further comprising at least one inhibitor of glutamine metabolism. These active substances can be administered simultaneously or sequentially. Glutamine is very important for tumor growth and its high consumption is typical for tumors. Glutamine is used to generate energy and serves as a source of nitrogen for the synthesis of nucleic acids and several amino acids. It is also involved in the regulation of cellular redox homeostasis. Glutamate produced from glutamine through glutaminase activity is further metabolized to alpha-ketoglutarate and thus provides a carbon source for a very important cycle of tricarboxylic acids.

TLR ligands are substances responsible for the infiltration of tumors by innate immune cells and for their activation. Furthermore, TLR ligands contribute to the formation of the Th1 anti-tumor environment and promote the formation of co-stimulatory molecules. In this way, TLR ligands promote efficient antigen presentation and the involvement of adaptive immunity. In a preferred embodiment, the TLR ligand is a substance selected from the group comprising resiquimod (R-848), polyinosinic: polycytidylic acid in the form of free acid or in salt form (poly (I:C)), lipoteichoic acid in the form of free acid or in salt form (LTA); more preferably a combination of at least two of those substances. Acid salts herein designate salts of an acid and a pharmaceutically acceptable cation, in particular alkali metal cations, alkaline earth metal cations, and ammonium cations.

In a preferred embodiment, the compounds labelling tumor cells as the target of innate immune cell attack are mainly ligands of cell phagocytic receptors, such as ligands of dectin-1, MR, MBL, CR3, CR4 receptors and scavenger receptors SR-A1, SR-A2 and MARCO. The compound that labels tumor cells as the target of innate immune cell attack is preferably a mannan-biocompatible anchor for membrane (mannan-BAM) conjugate. These compounds bind to tumor cells and label them as targets of innate immune cell attack. Innate immunity cells include neutrophils, monocytes, macrophages, dendritic cells, and NK cells.

Anti-CD40 antibody promotes both innate and adaptive immunity.

In a preferred embodiment, the inhibitors of glutamine metabolism are selected from the group comprising azaserine, 6-diazo-5-oxo-norleucine called DON, 5-diazo-4-oxo-L-norvaline called L-DONV, acivicin, azotomycin, bis-2-(5-phenylacetamido-1,3,4-thiadiazol-2-yl)ethyl sulphide called BPTES, ebselen, chelerythrine, apomorphine, 2-(pyridin-2-yl)-N-(5-(4-(6-(2-(3-(trifluoromethoxy)phenyl)acetamido (pyridazin-3-yl)butyl)-1,3,4-thiadiazol-2-yl)acetamide called CB-839. These substances may be administered in the form of their prodrugs. The prodrugs react to form an active substance in the organism. The prodrugs are also included herein within the term “inhibitor of glutamine metabolism”.

Preferably, the present invention provides a pharmaceutical combination comprising resiquimod, polyinosinic: polycytidylic acid in the form of free acid or in salt form, lipoteichoic acid in the form of free acid or in salt form, mannan-BAM, an anti-CD40 antibody, and at least one inhibitor of glutamine metabolism.

In a preferred embodiment, the present invention provides a pharmaceutical combination comprising resiquimod, polyinosinic: polycytidylic acid in the form of free acid or in salt form, lipoteichoic acid in the form of free acid or in salt form, mannan-BAM, an anti-CD40 antibody, and 6-diazo-5-oxo-norleucin or a prodrug thereof.

In a preferred embodiment, the pharmaceutical combination is administrated intraperitoneally, subcutaneously or intratumorally into the tissue where tumor is located.

The object of the invention is further achieved by the use of the pharmaceutical composition for treatment of cancer, in particular treatment of solid tumors, most preferably treatment of pancreatic adenocarcinoma, comprising a step of administering a pharmaceutical combination of active substances for immunotherapy comprising at least one TLR (Toll-like receptor) ligand, at least one compound that labels tumor cells as the target of immune cell attack, at least one anti-CD40 antibody, and further at least one inhibitor of glutamine metabolism to a human or animal subject in need of such treatment.

In a preferred embodiment, the pharmaceutical combination of active substances comprising of resiquimod, polyinosinic: polycytidylic acid, lipoteichoic acid, mannan-BAM, an anti-CD40 antibody is administered intratumorally, and prior to the said mixture is administrated 6-diazo-5-oxo-L-norleucin intratumorally or systemically.

In a preferred embodiment, the pharmaceutical combination of active substances for immunotherapy comprising at least one TLR (Toll-like receptor) ligand, at least one compound that labels tumor cells as the target of immune cell attack, and at least one anti-CD40 antibody is administered intratumorally, and subsequently to the said mixture is administrated at least one inhibitor of glutamine metabolism.

In a preferred embodiment, the pharmaceutical combination of active substances comprising of resiquimod, polyinosinic: polycytidylic acid, lipoteichoic acid, mannan-BAM, an anti-CD40 antibody is administered intratumorally, and subsequently to the said mixture is administrated 6-diazo-5-oxo-L-norleucin intratumorally or systemically.

In a preferred embodiment, the pharmaceutical combination of active substances for immunotherapy comprising at least one TLR ligand, at least one compound labelling tumor cells as the target of immune cell attack, at least one anti-CD40 antibody and at least one inhibitor of glutamine metabolism are administrated simultaneously.

In a preferred embodiment, the pharmaceutical combination of active substances comprising of resiquimod, polyinosinic: polycytidylic acid, lipoteichoic acid, mannan-BAM, an anti-CD40 antibody is administered intratumorally, and simultaneously is administrated 6-diazo-5-oxo-L-norleucin intratumorally or systemically.

The substances used in the present invention are commercially available. Although the individual substances or their sub-combinations are already known in the art, it has now surprisingly been found that the combination of these active substances, i.e., immunometabolic treatment based on the involvement of the corresponding mechanisms of actions, has a synergistic effect, causing a tumor to stop growing or even shrink and increase survival even in large and advanced solid tumors.

The advantage of the pharmaceutical combination for use in the treatment of cancer is that it results in the complete elimination of advanced subcutaneous tumor if used in treatment and furthermore provides long-term immune memory.

### BRIEF DESCRIPTION OF DRAWINGS

The present invention will be explained in detail by means of the following figures where:

FIG. 1 shows the results of immunometabolic treatment of mice with pancreatic adenocarcinoma using the pharmaceutical combination, FIGS. 1.A and B show reduction of tumor growth, FIG. 1.C shows prolongation of survival of mice,

FIG. 2 shows the results of immunometabolic treatment of mice with very advanced pancreatic adenocarcinoma using the pharmaceutical combination, FIG. 2.A shows reduction of tumor growth, FIG. 2.B shows prolongation of survival of mice,

FIG. 3 shows the results of immunometabolic treatment of mice with pancreatic adenocarcinoma using the pharmaceutical combination, FIG. 3.A show reduction of tumor growth, FIG. 3.B shows prolongation of survival of mice,

FIG. 4 shows the results of the pharmaceutical combination of intratumoral MBTA therapy and i.p. application of glutamine antagonist (DON) in two-timing regimes in bilateral Panc02 model, FIGS. 4.A and B show reduction of tumor growth, FIG. 4.C shows prolongation of survival of mice,

FIG. 5 shows the results of s.c. application of DON in different dosages in bilateral Panc02 model, FIGS. 5.A and B show reduction of tumor growth, FIG. 5.C shows prolongation of survival of mice,

FIG. 6 shows the results of comparison of i.t. and i.p. routes of DON application in bilateral Panc02 model, FIGS. 6.A and B show reduction of tumor growth, FIG. 6.C shows prolongation of survival of mice,

FIG. 7 shows the results of combination of MBTA immunotherapy and glutamine metabolism inhibition by DON in bilateral Panc02 model-evaluation of side effects, FIGS. 7.A and B show reduction of tumor growth, FIG. 7.C shows prolongation of survival of mice, FIG. 7.D shows food consumption, FIG. 7.E shows water consumption, FIG. 7.F shows weight of mice,

FIG. 8 shows the effect of DON on immune cell infiltration of left MBTA untreated parallel pancreatic adenocarcinoma tumors, Bilateral Panc02 model.

### PREFERRED EMBODIMENTS OF THE INVENTION

### Pharmaceutical Combination

Pharmaceutical combination further described for testing its activity in a method of cancer treatment, especially solid tumor treatment as pancreatic adenocarcinoma is, contained two TLR ligands in form of sodium salt of polyinosinic: polycytidyl acid (poly (I:C)) and lipoteichoic acid, anti-CD40 antibody, one compound labelling tumor cells in form of mannan-BAM and one inhibitor of glutamine metabolism in form of 6-diazo-5-oxo-norleucin or called DON. In another not shown embodiment the pharmaceutical combination comprises of at least one TLR ligand, preferably at least two, selected from the group of resiquimod, polyinosinic: polycytidylic acid in form of acid or in salt form, lipoteichoic acid in the form of the acid or in salt form. In another not shown embodiment the pharmaceutical combination comprises of at least one compound labelling tumor cells selected from the group of dectin-1, mannose receptor, mannan binding lectin, complement receptor 3, complement receptor 4, and scavenger receptors class A ligands. A larger number of these phagocytosis-stimulating ligands offers the possibility of selecting molecules optimal in terms of penetration through the tumor environment and in terms of anchoring to the surface of tumor cells. In another not shown embodiment the pharmaceutical combination comprises of at least one inhibitor of glutamine metabolism selected from the group of azaserine, 5-diazo-4-oxo-L-norvalin, acivicin, azotomycin, bis-2-(5-phenylacetamido-1,3,4-thiadiazol-2-yl)ethyl sulfide, ebselen, chelerythrine, apomorphine, 2-(pyridin-2-yl)-N-(5-(4-(6-(2-(3-(trifluoromethoxy)phenyl)acetamido) pyridazin-3-yl)butyl)-1,3,4-thiadiazol-2-yl)acetamid. The range of potential inhibitors of glutamine metabolism allows the identification of a substance with a wide therapeutic window, i.e. the separation of therapeutically optimal concentrations and concentrations causing side effects.

### Materials

Tissue culture media, culture media supplements, mannan from Saccharomyces cerevisiae, lipoteichoic acid (LTA) from Bacillus subtilis, and sodium salt of polyinosinic: polycytidyl acid (poly (I:C)) were obtained from Sigma-Aldrich (St. Louis, MO, USA). Resiquimod (R-848) was supplied by Tocris Bioscience (Bristol, UK). The Biocompatible Anchor for cell Membrane (BAM, Mw 4000) was obtained from NOF EUROPE (Grobbendonk, Belgium). Monoclonal antibody anti-CD40 (rat IgG2a, clone PGK4.5/PGK45) was supplied by BioXCell (West Lebanon, NH, USA). 6-diazo-5-oxo-L-noreucine (DON) was obtained from Institute of Organic Chemistry and Biochemistry (ÚOCHB), Prague; however, it can also be purchased from commercial suppliers.

### Cell Lines and Mice

The mouse pancreatic adenocarcinoma line Panc02 was donated by Prof. Lars Ivo Parteck (Greifswald, Germany). Cells were maintained in Dulbecco's modified Eagle medium (DMEM) supplemented with 10% heat-inactivated fetal bovine serum and antibiotics (PAA, Pasching, Austria). The cells were cultured at 37° C. in an atmosphere saturated with water vapour and containing 5% carbon dioxide.

Specific pathogen-free C57BL/6 female mice or called SPF C57BL/6 mice (females) were obtained from Charles River Laboratories (Sulzfeld, Germany). Mice weighting between 18 and 20 g were housed in a barrier facility in a specific pathogen-free environment with free access to sterile food and water; the photoperiod was 12/12.

### Synthesis of Mannan-BAM

The synthesis of mannan-BAM was performed as previously described (Janotová T, Jalovecká M, Auerova et al., The use of anchored agonists of phagocytic receptors for cancer immunotherapy: B16-F10 murine melanoma model, PLOS ONE, p 1-14, 2014).

### Tumor Transplantation

Subcutaneous transplantation: Mice were subcutaneously injected with 4×10⁵ Panc02 cells in 0.1 ml DMEM or 4×10⁵ B16-F10 cells in 0.1 ml RPMI 1640 medium without additives into pre-shaved groin (right or both right and left).

Intracranial transplantation: Mice were first anesthetized by intraperitoneal injection of ketamine (Narkamon, Bioveta, Czech Republic, 100 mg/kg) and xylazine (Rometar, Bioveta, Czech Republic, 5 mg/kg) mixture. Subsequently, mice were injected intracranially with 1×10⁵ Panc02 cells in 0.03 ml DMEM without additives.

### Treatment and its Evaluation

MBTA immunotherapy: 50 μl of the therapeutic mixture consisting of 0.5 mg R-848, HCl form+0.5 mg poly (I:C), 0.5 mg LTA and 0.4 mg anti-CD40/ml of 0.2 mM mannan-BAM in PBS was administered intratumorally on days 0, 1, 2, 8, 9, 10, 16, 17, 18, 24, 25, and 26. The combination of R-848, poly (I:C), LTA, mannan-BAM and anti-CD40 antibody is hereinafter referred to as ‘MBTA’. In the case of the bilateral Panc02 tumor model, MBTA therapy was always injected only into the right subcutaneous tumor.

DON metabolic intervention: 100 μl of DON solution (4 mg DON/ml in phosphate buffered saline so called PBS) was applied intraperitoneally (i.p.), as further specified. In Example 3, subcutaneous (s.c.) administration of 200 μl DON solution (4 mg DON/1 ml PBS) was also used. Further was studied intratumoral administration (i.t.) in further specified doses and schedules as outlined in examples.

Therapeutic vaccines: For the Panc02-MBTA vaccine, Panc02 cells were resuspended in DMEM media containing non-heat inactivated fetal bovine serum and 0.02 mM mannan-BAM. After 2 hours of incubation at 37° C., Panc02 cells were washed and resuspended in the therapeutic mixture consisting of 0.5 mg R-848 (HCl form), 0.5 mg poly (I:C), 0.5 mg LTA, and 0.4 mg anti-CD40 per ml of 0.2 mM mannan-BAM in PBS (4×106 Panc02 cells per 1 ml of the therapeutic mixture). For the Panc02-PBS vaccine, Panc02 cells were resuspended in PBS (4×106 Panc02 cells per 1 ml PBS). Subsequently, Panc02 cells for the Panc02-MBTA and the Panc02-PBS vaccine were killed by a freeze-thaw cycle, and the viability of the cells was determined by vital staining. 50 μl of vaccines were applied subcutaneously (s.c.) into the previously shaved lower dorsal site (right flank) in a pulse regime on the following days: 0, 1, 2, 8, 9, 10, 16, 17, 18, 24, 25, and 26.

Evaluation of treatment: Tumor sizes were measured every other day using a digital calliper. The formula V=(π/6)AB², A being the largest tumor size, B being the smallest tumor size, was used to calculate tumor volume.

### Flow Cytometry Analysis of Tumor-Infiltrating Leukocytes

Analysis of tumor-infiltrating leukocytes was performed as described in Caisová V, Uher O, Nedbalová P et al., Effective cancer immunotherapy based on combination of TLR agonists with stimulation of phagocytosis, Int Immunopharmacol., p. 86-96, 2018. In brief, tumors were digested with Liberase DL and Dnase I (both Roche Diagnostics, Germany). The following monoclonal antibodies (eBioscience, San Diego, CA, USA) were used: a) total leukocytes-anti-mouse CD45 PerCP-Cy5.5, clone 30-F11; b) T lymphocytes-anti-mouse CD3e FITC, clone 145-2C11; c) CD4+ T lymphocytes-anti-mouse CD4 APC, clone GK1.5; d) CD8+ T lymphocytes-anti-mouse CD8a, clone 53-6.7; e) B lymphocytes-anti-mouse CD19 APC, clone eBio1D3; f) NK cells-anti-mouse NK1.1 PE, clone PK136; g) granulocytes-anti-mouse Gr-1 Alexa Fluor 700, clone RB68C5; and h) macrophages-anti-mouse F4/80 Antigen PE-Cy7, clone BM8. Analysis was performed using a BD FACSCanto Il flow cytometer (BD Biosciences, San Jose, CA, USA) and BD FACSDiva software 6.1.3. (BD Biosciences, San Jose, CA, USA).

### Statistical Analysis

The areas under the curves (AUC) were calculated to evaluate tumor growth. The obtained values were statistically evaluated by one-way ANOVA with unequal post hoc test. Kaplan-Meier survival curves were evaluated using the long-rank test. STATISTICA 12 software (StatSoft, Inc., Tulsa, OK, USA) was used for all statistical evaluations. Error bars represent standard error of the mean (SEM).

### Example 1: Synergy of MBTA Immunotherapy with Inhibition of Glutamine Metabolism by DON—Two-Tumor Mouse Model of Pancreatic Adenocarcinoma (Panc02)

The pharmaceutical combination has been tested for the treatment of pancreatic adenocarcinoma, which is considered to belong among the worst oncological diseases. Mice with two advanced tumors were treated by intratumoral administration of MBTA to one (right) tumor simultaneously with intraperitoneal administration of a glutamine metabolism inhibitor, in this case was used 6-diazo-5-oxo-norleucin or called DON.

C57BL/6 mice were inoculated subcutaneously with 4×10⁵ mouse pancreatic adenocarcinoma Panc02 cells in 0.1 ml DMEM in the shaved area of the right and left groin. Fourteen days after tumor cell transplantation when the average primary tumor volume was 100.7±42.1 mm³, and the distant tumor was 104.8±43.2 mm³, mice were randomized into groups of six and therapies were initiated immediately. MBTA mixture (PBS in control) was applied intratumorally (50 μl MBTA mixture: 0.5 mg R-848+0.5 mg poly (I:C)+0.5 mg LTA+0.4 mg anti-CD40/ml 0.2 mM mannan-BAM in PBS) in pulse mode (days 0, 1, 2, 8, 9, 10, 16, 17, 18, 24, 25, 26) to right tumors. DON (PBS in control) was applied intraperitoneally (100 μl solution 4 mg DON/ml PBS) on days 5, 13, 21, 29, 37.

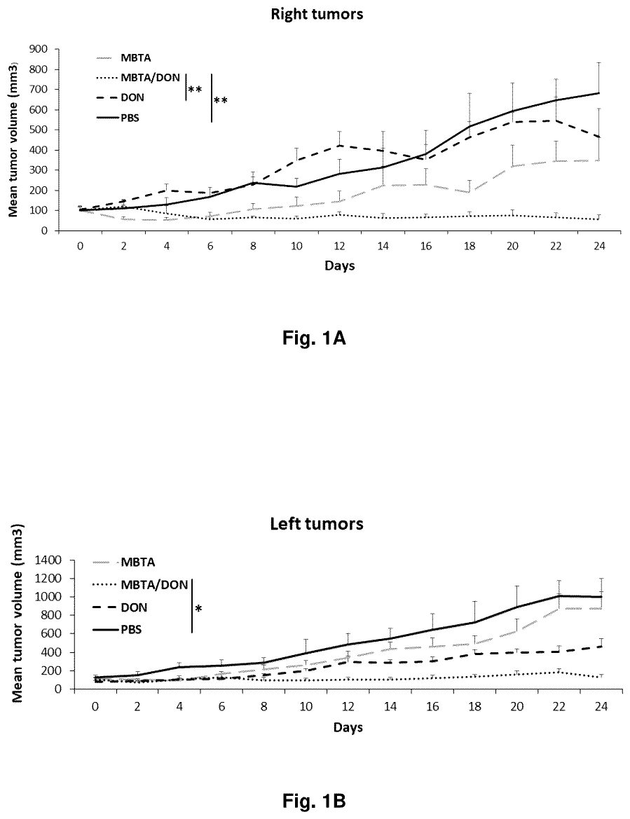

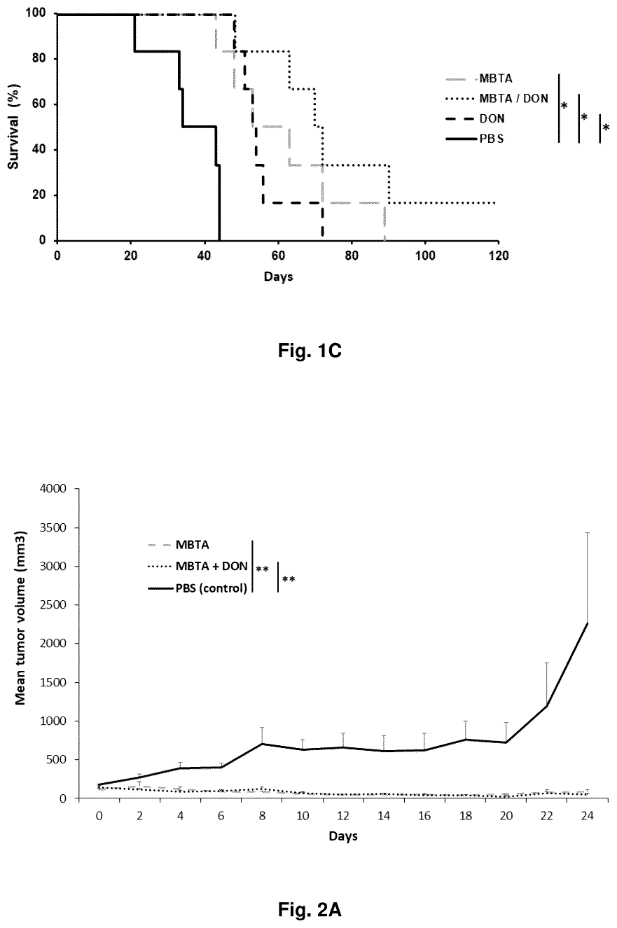

Immunometabolic combination MBTA and DON showed synergy in both reducing tumor growth (FIG. 1A, B) and prolonging mouse survival (FIG. 1C) compared to other groups where none of mice remained tumor free. This experiment was performed twice with comparable results.

FIG. 1A, B: Growth of right/left tumors. Statistical analysis was performed by calculating AUC (area under the curve) values using a one-way ANOVA test with Tukey's post hoc test, *=p<0.05 **=p<0.01, FIG. 1 C: Survival analysis, long rank test, *=p<0.05.

### Example 2: Synergy of MBTA Immunotherapy with Inhibition of Glutamine Metabolism by DON-Very Advanced Pancreatic Adenocarcinoma (Panc02), Mouse Model

The pharmaceutical combination has been tested for the treatment of very advanced pancreatic adenocarcinoma.

C57BL/6 mice were inoculated subcutaneously with 4×10⁵ mouse pancreatic adenocarcinoma cells Panc02 in 0.1 ml DMEM in the shaved area of the right groin. Twenty days after tumor cell transplantation when the tumors reached large dimensions (140.8+46.8 mm³, range 37.4 to 247.0 mm³) mice were randomized into groups of six and appropriate therapies were initiated immediately. MBTA mixture (PBS in control) was applied intratumorally (50 μl MBTA mixture: 0.5 mg R-848+0.5 mg poly (I:C)+0.5 mg LTA+0.4 mg anti-CD40/ml 0.2 mM mannan-BAM in PBS) in pulse mode (days 0, 1, 2, 8, 9, 10, 16, 17, 18, 24, 25, 26). DON was applied intraperitoneally (100 μl solution 4 mg DON/ml PBS) on days 0, 7, 14, 21, 28.

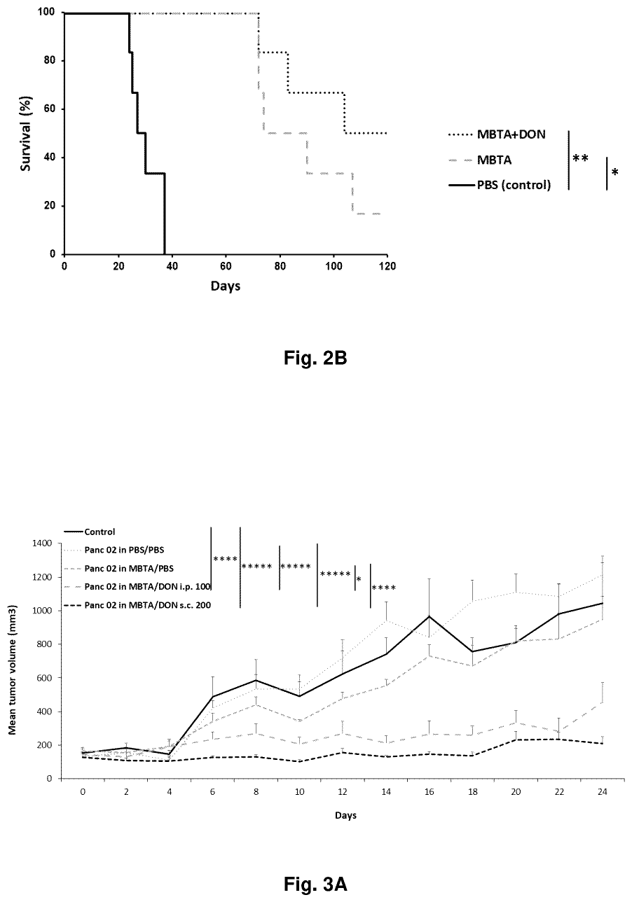

The reduction of tumor growth in mice treated only with MBTA therapy and MBTA therapy together with DON were similar (FIG. 2A). However, the addition of DON to MBTA therapy resulted in the improved survival of mice (3/6) compared to mice treated only with MBTA therapy where only one mouse (1/6) remained tumor-free (FIG. 2 B).

Moreover, three mice surviving from MBTA/DON group were subsequently intracranially injected with 1×10⁵ Panc02 cells (intracranial injections were performed after 174 days from the primary inoculation of Panc02 cells). All three mice were fully resistant to this re-transplantation. In contrast, all mice (n=8) in the control group died in 11-14 days after the Panc02 intracranial transplantation (data not shown). The resistance to the intracranial tumor transplantation demonstrated the ability of this combined therapy to induce systemic, long-lasting immunological memory also in the immune-privileged site such as the central nervous system.

FIG. 2A: Growth of tumors. Statistical analysis was performed by calculating AUC (area under the curve) values using a one-way ANOVA test with Tukey's post hoc test, **=p<0.01, FIG. 1 B: Survival analysis, long rank test, *=p<0.05 **=p<0.01.

### Example 3: Synergy of MBTA Immunotherapy with Inhibition of Glutamine Metabolism by DON—Pancreatic Adenocarcinoma (Panc02), Mouse Model, Vaccination

The pharmaceutical combination has been tested for the treatment of pancreatic adenocarcinoma. Mice with advanced left tumors were injected subcutaneously (right flank) with a Panc02 dead cell suspension in MBTA, concomitantly with intraperitoneal/subcutaneous administration of the glutamine metabolism inhibitor DON.

C57BL/6 mice were inoculated subcutaneously with 4×10⁵ mouse pancreatic adenocarcinoma cells Panc02 in 0.1 ml DMEM in the shaved area of the left groin. Fourteen days after tumor transplantation, mice were randomized into groups of six and appropriate therapies were initiated immediately. Suspension of frost-killed Panc02 cells in MBTA (PBS in control) was applied subcutaneously to the shaved area of the right groin (50 μl MBTA/PBS suspension: 4 mil dead Panc02 cells+0.5 mg R-848+0.5 mg poly (I:C)+0.5 mg LTA+0.4 mg anti-CD40/ml 0.2 mM mannan-BAM in PBS) in pulse mode (days 0, 1, 2, 8, 9, 10, 16, 17, 18, 24, 25, 26). DON was applied intraperitoneally (i.p. 100 μl solution 4 mg DON/ml PBS) and subcutaneously (s.c. 200 μl solution 4 mg DON/ml PBS), both on days 5, 13, 21, 29, 37.

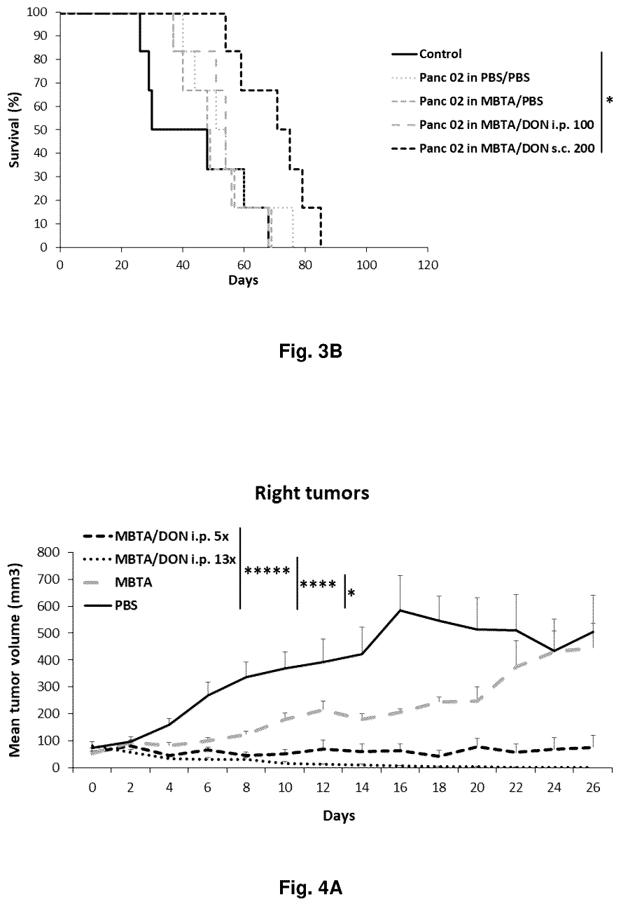

The pharmaceutical combination MBTA and DON showed synergy in reducing tumor growth (FIG. 3A) and in the case of subcutaneous application of DON, synergy in prolonging the survival of mice was also observed (FIG. 3 B).

FIG. 3A: Growth of tumors. Statistical analysis was performed by calculating AUC (area under the curve) values using a one-way ANOVA test with Tukey's post hoc test, *=p<0.05 ****=p<0.001 *****=p<0.0005, FIG. 3 B: Survival analysis, long rank test, *=p<0.05.

### Example 4: Combination of Intratumoral MBTA Therapy and i.p. Application of Glutamine Antagonist (DON) in Two-Timing Regimes in Bilateral Panc02 Model

To improve MBTA/DON combined therapy outcomes we tested the intraperitoneal administration of DON in combination with i.t. MBTA therapy. The total amount of DON (2 mg/mouse/therapy) was applied in two different regimes as further specified.

C57BL/6 mice were s.c. inoculated with Panc02 cells in both right and left flank. After 12 days, mice were randomized into four groups (n=6/group): Group treated with PBS applied in the right tumor; group treated with MBTA applied in the right tumor; group treated with MBTA applied in the right tumor, and DON applied i.p. (13 doses); group treated with MBTA applied in the right tumor, and DON applied i.p. (5 doses, red). MBTA (PBS in the control group) was applied in pulse regime on days 0, 1, 2, 8, 9, 10, 16, 17, 18, 24, 25, and 26. DON was administered i.p. on days 5, 13, 21, 29, 37 (400 μg) and 1, 3, 5, 7, 9, 11, 13 15 17 19 21, 23, 25 (154 μg), respectively.

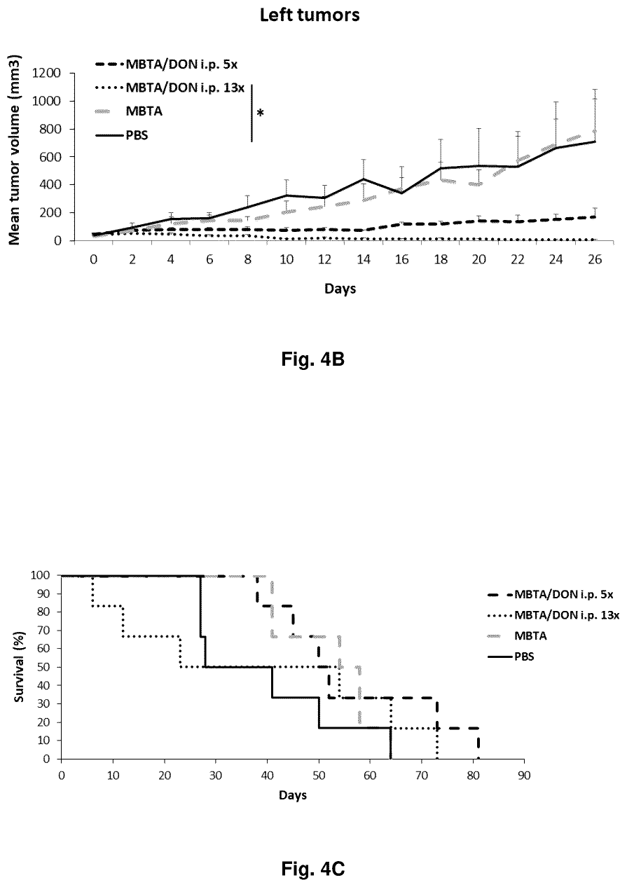

As shown in FIG. 4A, B both regimes of DON application had similar effect on the reduction of tumor growth. The prolongation of mice survival in both regimes (FIG. 4 C) was evident but not statistically significant. However, the second regime (application of DON every other day), was found to be too aggressive and resulted in early death of three mice.

FIG. 4A, B: Growth of right/left tumors. Statistical analysis was performed by calculating AUC (area under the curve) values using a one-way ANOVA test with Tukey's post hoc test (* p<0.05, **** p<0.001, ***** p<0.0005), FIG. 4 C: Survival analysis.

### Example 5: S.c. Application of DON in Different Dosages in Bilateral Panc02 Model

Subcutaneous administration of DON was tested in combination with i.t. MBTA therapy. DON was tested in two different concentration regimes. In the first regime, the same amount of DON as in Example 4 (2 mg/mouse/therapy) was applied in 5 subcutaneous injections parallel with MBTA therapy (400 μg/mouse/injection). The second regime was based on lower doses (80 μg/mouse/injection) with the same timing.

C57BL/6 mice were s.c. inoculated with Panc02 cells to both the right and left flank. After 12 days, mice were randomized into four groups (n=6/group): Group treated with PBS applied in the right tumor; group treated with MBTA applied in the right tumor; group treated with MBTA applied in the right tumor, and low doses of DON applied s.c.; group treated with MBTA applied in the right tumor and high doses of DON applied s.c. MBTA (PBS in the control group) was applied in pulse regime on days 0, 1, 2, 8, 9, 10, 16, 17, 18, 24, 25, and 26. DON (doses 80 μg and 400 μg, respectively) was applied s.c. on days 1, 9, 17, 25, 33.

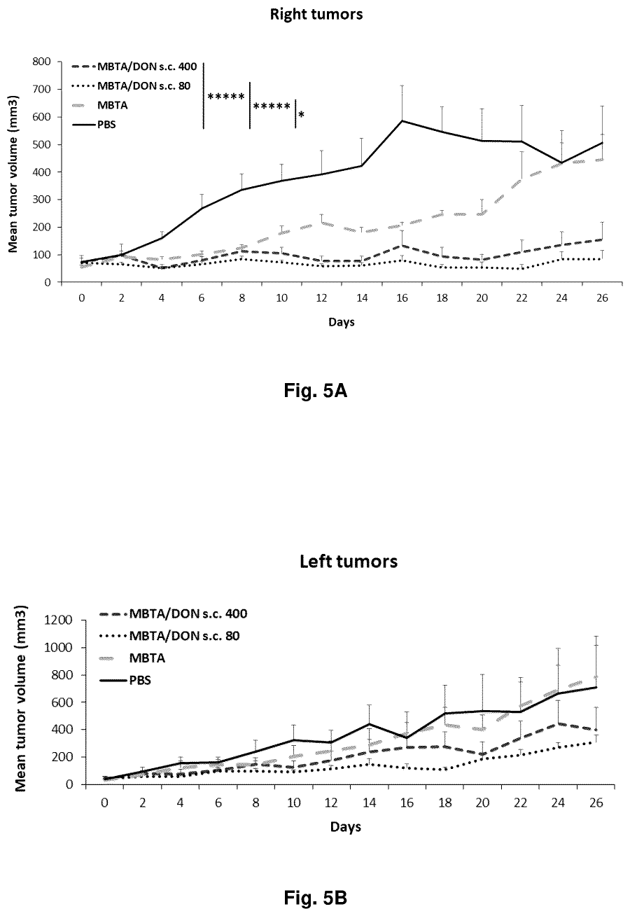

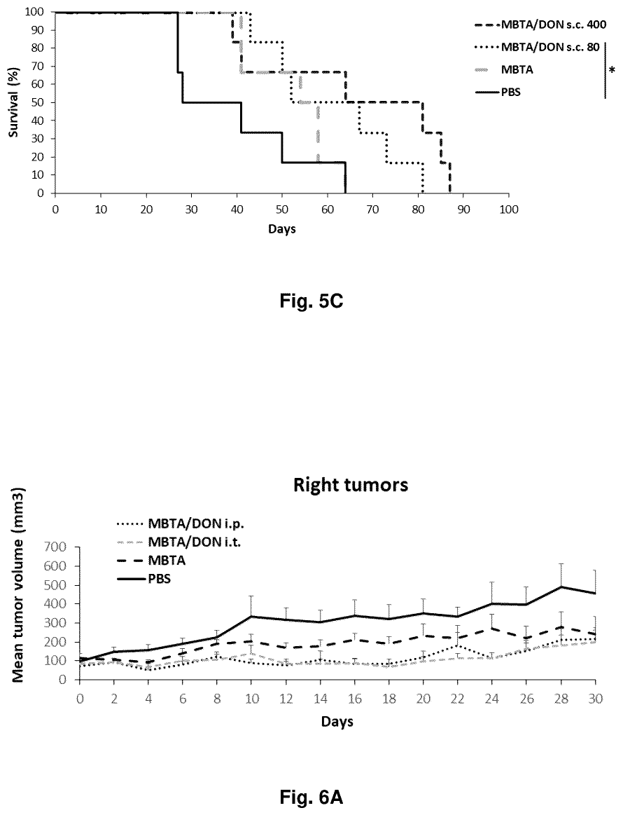

As shown in FIG. 5A, B, the reduction of tumor growth was less evident compared to i.p. administration of DON (Example 4, FIG. 4A, B). No early deaths of treated mice were observed (FIG. 5 C).

FIG. 5A, B: Growth of right/left tumors. Statistical analysis was performed by calculating AUC (area under the curve) values using a one-way ANOVA test with Tukey's post hoc test (* p<0.05, ***** p<0.0005), FIG. 5 C: The survival curve analysis (* p<0.05).

### Example 6: Comparison of i.t. And i.p. Routes of DON Application in Bilateral Panc02 Model

Two schemes were compared, first DON was injected into distal MBTA-non-treated tumors, and second, DON was given intraperitoneally.

C57BL/6 mice were s.c. inoculated with Panc02 cells in both, right and left flank. After 12 days, mice were randomized into four groups (n=6/group): Group treated with PBS applied in the right tumor; group treated with MBTA applied in the right tumor; group treated with MBTA applied in the right tumor, and DON applied in left tumor; group treated with MBTA applied in the right tumor, and DON applied i.p. MBTA (PBS in the control group) was applied in pulse regime on days 0, 1, 2, 8, 9, 10, 16, 17, 18, 24, 25, and 26. DON (400 μg) was applied i.t. or i.p. on days 1, 8, 15, 22, 29.

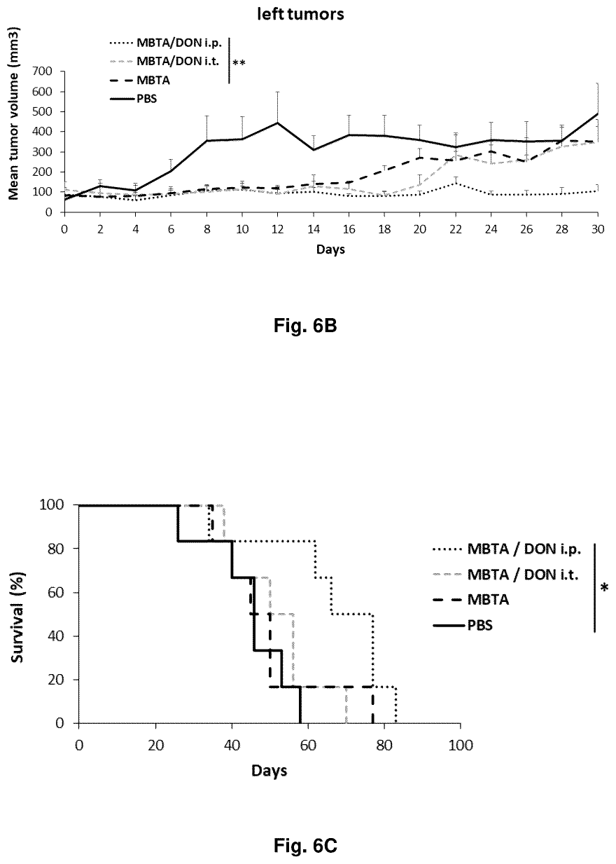

Intratumoral application of DON did not improve the efficacy of the therapy in contrast to its i.p. application that significantly prolonged the survival of treated mice (FIG. 6A, B, C)

FIG. 6A, B: Growth of right/left tumors. Statistical analysis was performed by calculating AUC (area under the curve) values using a one-way ANOVA test with Tukey's post hoc test (** p<0.01, Kruskal-Wallis test), FIG. 6 C: The survival curve analysis (* p<0.05).

### Example 7: Combination of MBTA Immunotherapy and Glutamine Metabolism Inhibition by DON in Bilateral Panc02 Model—Evaluation of Side Effects

Examples 4 to 6 have shown that the optimal way to apply DON is by i.p. injection with a sufficient interval between each application. Although, we did not observe any early deaths of treated mice in this regime, we wanted to confirm that this administration does not influence wellbeing of treated mice.

C57BL/6 mice were s.c. inoculated with Panc02 cells to both the right and left flank. After 12 days, mice were randomized into three groups (n=6/group): Group treated with PBS applied in the right tumor; group treated with MBTA applied in the right tumor; group treated with MBTA applied in the right tumor, and DON applied i.p. MBTA (PBS in the control group) was applied in the pulse regime on days 0, 1, 2, 8, 9, 10, 16, 17, 18, 24, 25, and 26. DON (400 μg) was applied i.p. on days 1, 8, 15, 22, 29.

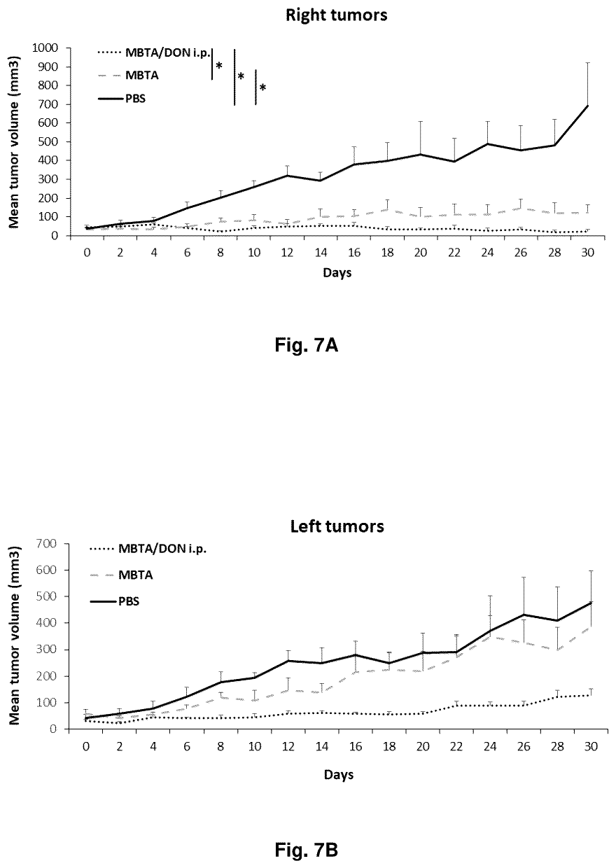

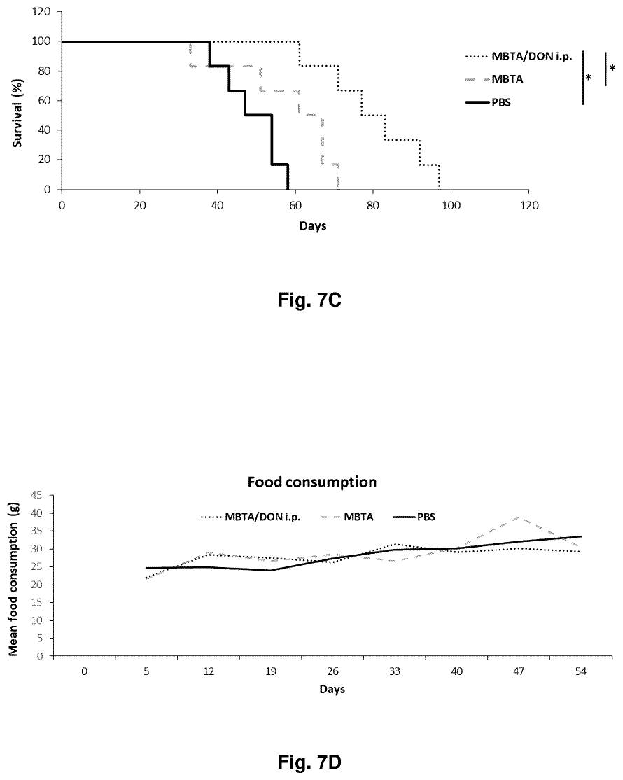

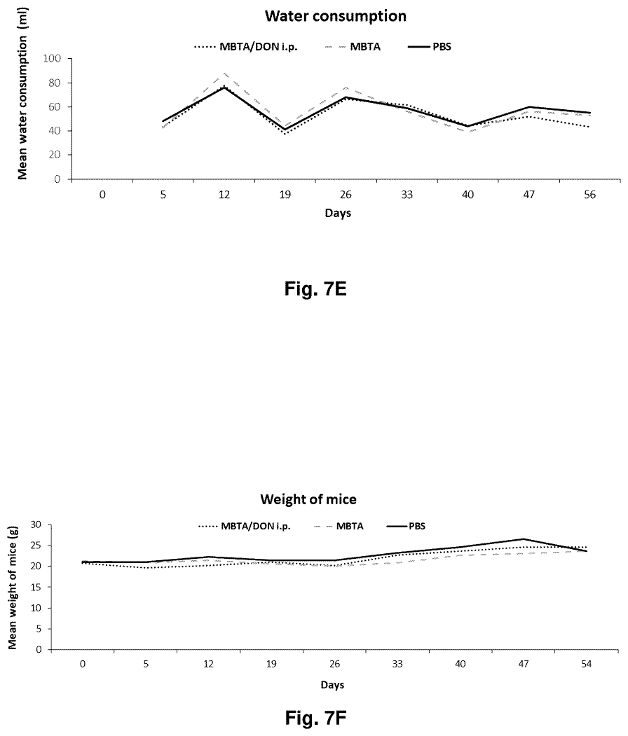

Combination of MBTA therapy and i.p. DON application resulted in reduction of growth of both tumors and significant prolongation survival of treated mice (FIG. 7A-C) without any influence of DON administration on water and food consumption, as well as weight of treated mice, as shown in FIG. 7 D-F.

FIG. 7A, B: Growth of right/left tumors. Statistical analysis was performed by calculating AUC (area under the curve) values using a one-way ANOVA test with Tukey's post hoc test (* p<0.05), FIG. 7 C: The survival curve analysis (* p<0.05), FIG. 7 D, E, F: Evaluation of food and water consumption and weight changes of mice during the experiment.

### Example 8: The Effect of DON on Immune Cell Infiltration of Left MBTA Untreated Parallel Pancreatic Adenocarcinoma Tumors, Bilateral Panc02 Model

Considering possible side effects of DON administration, the question of whether DON has a negative impact on the infiltration of a distal tumor was considered.

C57BL/6 mice were s.c. inoculated with Panc02 cells in both right and left flank. After 14 days, mice were randomized into four groups (n=16/group): Group treated with PBS applied in the right tumor and PBS i.p.; group treated with MBTA applied in the right tumor and PBS i.p.; group treated with PBS applied in the right tumor and DON applied i.p.; group treated with MBTA applied in right tumor and DON applied i.p. MBTA (PBS in the control group) was applied in the pulse regime on days 0, 1, 2, 8, 9, 10, 16, 17, 18, 24, 25, and 26. DON (400 μg) was applied i.p. on days 5, 13, 21, 29. Flow cytometry analysis was performed on days 7, 15, 23, 31.

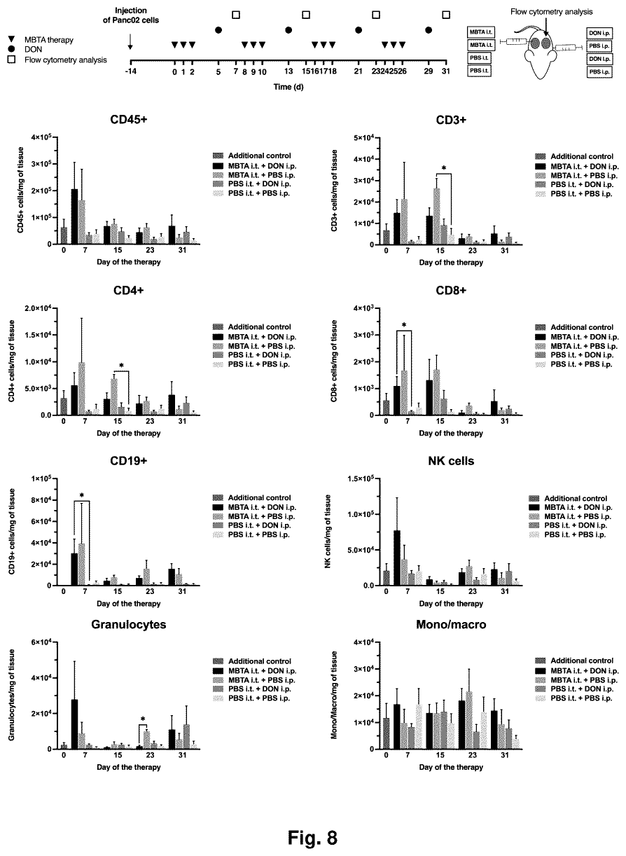

As shown in FIG. 8 with only one exception (number of granulocytes on day 23), there was not observed any significant changes connected to the addition of DON to MBTA therapy.

FIG. 8: The effect of DON application on immune cell infiltration of left MBTA untreated parallel pancreatic adenocarcinoma tumors in bilateral Panc02 model.

### INDUSTRIAL APPLICABILITY

A pharmaceutical combination for use in the treatment of cancer according to this invention can be used in treatment of cancer, especially for treatment of solid tumors as metastatic murine pancreatic adenocarcinoma is.

1. A pharmaceutical combination of active substances for use in a method of cancer treatment, comprising at least one TLR ligand, at least one compound labelling tumor cells as the target of immune cell attack, at least one anti-CD40 antibody, and further at least one inhibitor of glutamine metabolism.

2. The pharmaceutical combination according to claim 1, wherein the TLR ligand is a substance selected from the group comprising resiquimod, polyinosinic: polycytidylic acid in the form of the acid or in salt form, lipoteichoic acid in the form of the acid or in salt form.

3. The pharmaceutical combination according to claim 1, wherein the TLR ligand is a combination of at least two substances selected from resiquimod, polyinosinic: polycytidylic acid in the form of the acid or in salt form, lipoteichoic acid in the form of the acid or in salt form.

4. The pharmaceutical combination according to claim 1, wherein the compound labelling tumor cells as the target of the attack of innate immune cells is a ligand of cell phagocytic receptors selected from the group consisting of ligands of dectin-1, MR, MBL, CR3, CR4 receptors and scavenger receptors SR-A1, SR-A2 and MARCO or mannan-BAM.

5. The pharmaceutical combination according to claim 1, wherein the compound labelling tumor cells as the target of the attack of innate immune cells is mannan-BAM.

6. The pharmaceutical combination according to claim 1, wherein the inhibitor of glutamine metabolism is selected from the group comprising azaserine, 6-diazo-5-oxo-L-norleucin, 5-diazo-4-oxo-L-norvalin, acivicin, azotomycin, bis-2-(5-phenylacetamido-1,3,4-thiadiazol-2-yl)ethyl sulfide, ebselen, chelerythrine, apomorphine, 2-(pyridin-2-yl)-N-(5-(4-(6-(2-(3-(trifluoromethoxy)phenyl)acetamido) pyridazin-3-yl)butyl)-1,3,4-thiadiazol-2-yl)acetamid.

7. The pharmaceutical combination according to claim 1, wherein it comprises resiquimod, polyinosinic: polycytidylic acid in the form of free acid or in salt form, lipoteichoic acid in the form of free acid or in salt form, mannan-BAM, anti-CD40 antibody, and at least one inhibitor of glutamine metabolism.

8. The pharmaceutical combination according to claim 1, wherein it comprises resiquimod, polyinosinic: polycytidylic acid in the form of free acid or in salt form, lipoteichoic acid in the form of free acid or in salt form, mannan-BAM, anti-CD40 antibody, and 6-diazo-5-oxo-norleucin.

9. The pharmaceutical combination according to claim 1, wherein it is administered intraperitoneally, subcutaneously, or intratumorally into the tissue where tumor is located.

10. The pharmaceutical combination according to claim 1, wherein a mixture of active substances for immunotherapy comprising at least one TLR (Toll-like receptor) ligand, at least one compound that labels tumor cells as the target of immune cell attack, and at least one anti-CD40 antibody is administered intratumorally, and prior to the said mixture is administrated at least one inhibitor of glutamine metabolism.

11. The pharmaceutical combination according to claim 10, wherein a mixture of active substances comprising of resiquimod, polyinosinic: polycytidylic acid, lipoteichoic acid, mannan-BAM, an anti-CD40 antibody is administered intratumorally, and prior to the said mixture is administrated 6-diazo-5-oxo-L-norleucin intratumorally or systemically.

12. The pharmaceutical combination according to claim 1, wherein a mixture of active substances for immunotherapy comprising at least one TLR (Toll-like receptor) ligand, at least one compound that labels tumor cells as the target of immune cell attack, and at least one anti-CD40 antibody is administered intratumorally, and subsequently to the said mixture is administrated at least one inhibitor of glutamine metabolism.

13. The pharmaceutical combination according to claim 12, wherein a mixture of active substances comprising of resiquimod, polyinosinic: polycytidylic acid, lipoteichoic acid, mannan-BAM, an anti-CD40 antibody is administered intratumorally, and subsequently to the said mixture is administrated 6-diazo-5-oxo-L-norleucin intratumorally or systemically.

14. The pharmaceutical combination according to claim 1, wherein a mixture of active substances for immunotherapy comprising at least one TLR ligand, at least one compound labelling tumor cells as the target of immune cell attack, at least one anti-CD40 antibody and at least one inhibitor of glutamine metabolism are administrated simultaneously.

15. The pharmaceutical combination according to claim 14, wherein a mixture of active substances comprising of resiquimod, polyinosinic: polycytidylic acid, lipoteichoic acid, mannan-BAM, an anti-CD40 antibody is administered intratumorally, and simultaneously is administrated 6-diazo-5-oxo-L-norleucin intratumorally or systemically.

16. The use of the pharmaceutical combination according to claim 1 for cancer treatment.

17. The use according to claim 16, wherein the cancer is a solid tumor.

18. The use according to claim 16, wherein the cancer is pancreatic adenocarcinoma.
